(19) United States
(12) Patent Application Publication
(10) Pub. No.: US 2015/0225331 A1
(43) Pub. Date: Aug. 13, 2015

(54) METHOD FOR MANUFACTURING OLEFIN DERIVATIVE

(71) Applicant: JX NIPPON OIL & ENERGY CORPORATION, Tokyo (JP)

(72) Inventors: Daisuke Watanabe, Tokyo (JP); Akira Shiibashi, Tokyo (JP); Shin-ichi Komatsu, Tokyo (JP); Takaya Matsumoto, Tokyo (JP)

(73) Assignee: JX NIPPON OIL & ENERGY CORPORATION, Tokyo (JP)

(21) Appl. No.: 14/419,273

(22) PCT Filed: Jul. 8, 2013
(86) PCT No.: PCT/JP2013/068663

§ 371 Date: Feb. 3, 2015

(30) Foreign Application Priority Data
Aug. 9, 2012 (JP) 2012-177346

(51) Int. Cl. C07C 67/36; C07C 61/08

(52) U.S. Cl. CPC C07C 67/36; C07C 61/08; C07C 2103/94

(57) ABSTRACT

There is provided a method for manufacturing an olefin derivative which is capable of improving the yield and manufacturing efficiency. The method for manufacturing an olefin derivative according to the present invention comprises a first step of reacting an olefin with an alcohol and carbon monoxide in the presence of a palladium catalyst and an oxidizing agent in a reactor to thereby synthesize a carboxylic acid or a carboxylate ester, a second step of discharging at least part of a gas in the reactor out of the reactor, and separating carbon monoxide from the gas discharged during the first step; and a third step of supplying the carbon monoxide separated from the gas in the second step to the reactor during the first step.

### TECHNICAL FIELD

The present invention relates to a method for manufacturing olefin derivatives.

### BACKGROUND ART

As one example of methods for manufacturing olefin derivatives, a method is conventionally known which synthesizes carboxylic acids or carboxylate esters by an oxidative esterification reaction of olefins using carbon monoxide and an alcohol. In the oxidative esterification reaction, for the reproduction (oxidation) of a catalyst (for example, a Pd catalyst), an oxidizing agent (for example, CuCl₂) in an amount of not less than the equivalent of olefins is necessary. Therefore, the conventional manufacturing methods have a low efficiency of manufacturing the olefins, and are unsuitable for mass production (see, for example, Patent Literatures 1 and 2, and Non Patent Literatures 1 and 2 listed below). Further in the conventional manufacturing methods, after the reaction, target substances (olefin derivatives) need to be separated from an oxidizing agent and a compound (for example, CuCl) produced by the reduction of the oxidizing agent.

As one of the solutions to overcome these problems, there is proposed a method of using oxygen as an oxidizing agent (see, for example, Patent Literatures 3 and 4 listed below). In this method, oxygen turns to water after reproduction of a catalyst, and water can easily be separated from a product.

### CITATION LIST

### Patent Literature

Patent Literature 1: Japanese Patent Application Laid-Open No. 63-57589
Patent Literature 2: Japanese Patent Application Laid-Open No. 63-57557
Patent Literature 3: Japanese Patent Application Laid-Open No. 62-16448
Patent Literature 4: Japanese Examined Patent Publication No. 3342942 Non Patent Literature
Non Patent Literature 1: Journal of Molecular Catalyst A: Chemical 330(2010), 18-25
Non Patent Literature 2: New Journal of Chemistry 2002, 26, 387-397

### SUMMARY OF INVENTION

### Technical Problem

However, in the case of carrying out an oxidative esterification reaction using oxygen, usually, carbon monoxide, oxygen or air, and a raw material solution are charged in a pressure vessel and the reaction is allowed to proceed in the closed pressure vessel. In such a batch-type manufacturing method, steps must be carried out such as charging of a raw material to the pressure vessel, an oxidative esterification reaction, discharge of residual gas from the pressure vessel, and refinement of olefin derivatives for every one-time manufacture. For such a reason, even when the oxidative esterification reaction using oxygen is utilized, the manufacturing efficiency of the olefin derivatives is still low.

Additionally, part of carbon monoxide being the raw material gas does not react with olefins and is wasted as a residual gas, and olefins not having reacted with carbon monoxide resultantly remain. The yield and manufacturing efficiency of the olefin derivatives thereby decrease.

Further in the mass production of olefin derivatives by an oxidative esterification reaction using oxygen, since the pressures of carbon monoxide and oxygen in a pressure vessel decrease along with the progress of the reaction, these gases need to be replenished in the pressure vessel in the course of the reaction. When oxygen is supplied in a form of air to the pressure vessel, also nitrogen in the air is supplied together with oxygen to the pressure vessel. Nitrogen in the air, since not being consumed in the reaction, accumulates in the pressure vessel. As a result, the total pressure of the gases in the pressure vessel excessively rises, and there arises a fear that the total pressure reaches a tolerable limit value of the pressure vessel. In order to suppress a rise in the atmospheric pressure in the pressure vessel, instead of using air containing nitrogen, a high-purity oxygen alone in an amount corresponding to the amount of oxygen consumed in the pressure vessel needs to be replenished in the pressure vessel. However, since the high-purity oxygen is high in combustion supportability, there is a problem with the safety of a manufacturing method using the high-purity oxygen. If oxygen is diluted with nitrogen gas in order to reduce the combustion supportability of oxygen, the same problem as the above-mentioned problem in the case of using air occurs.

The present invention has been achieved in consideration of such problems in the conventional technologies, and has an object to provide a method for manufacturing olefin derivatives, the method being capable of improving the yield and manufacturing efficiency.

### Solution to Problem

In order to achieve the above-mentioned object, an aspect of a method for manufacturing an olefin derivative according to the present invention comprises a first step of reacting an olefin with an alcohol and carbon monoxide in the presence of a palladium catalyst and an oxidizing agent in a reactor to thereby synthesize a carboxylic acid or a carboxylate ester, a second step of discharging at least part of a gas in the reactor out of the reactor during the first step, and separating carbon monoxide from the gas discharged, and a third step of supplying the carbon monoxide separated from the gas in the second step to the reactor during the first step.

In an aspect of the present invention, the olefin may be an alicyclic olefin. The alicyclic olefin may be a norbornene-type compound.

In an aspect of the present invention, it is preferable that the gas discharged out of the reactor in the second step contains nitrogen.

In an aspect of the present invention, in the second step, carbon monoxide may be separated from the gas by using at least one method selected from the group consisting of a pressure swing adsorption method, a thermal swing adsorption method, a temperature-pressure swing adsorption method, a membrane separation method and a cryogenic separation method.

In an aspect of the present invention, at least one selected from the group consisting of oxygen, cupric acetate, cupric chloride, cupric nitrate, cupric sulfate, ferric chloride, ferric nitrate, ferric sulfate and ferric acetate may be used as the oxidizing agent.

### Advantageous Effects of Invention

According to the present invention, a method for manufacturing olefin derivatives, the method being capable of improving the yield and manufacturing efficiency, is enabled to be provided.

### BRIEF DESCRIPTION OF DRAWING

FIG. 1 is a schematic view of an apparatus for manufacturing olefin derivatives according to one embodiment of the present invention.

### DESCRIPTION OF EMBODIMENT

Hereinafter, by reference to the drawing, one preferred embodiment according to the present invention will be described in detail. However, the present invention is not limited to the following embodiment. Here, the same symbols are assigned to the same or equal elements. The ratios in dimension are not limited to those indicated in the drawing.

(Outline of a Method for Manufacturing Olefin Derivatives)

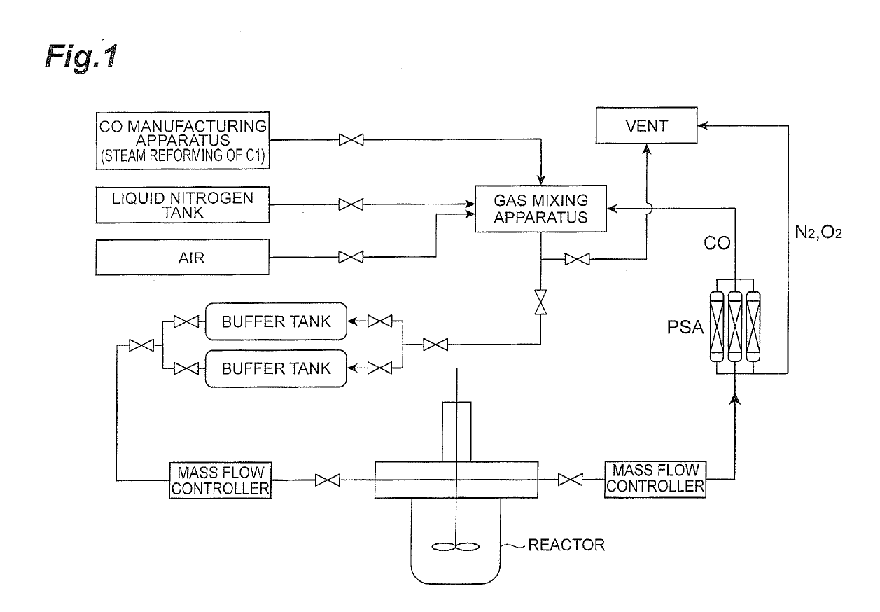

A method for manufacturing olefin derivatives according to the present embodiment is carried out using an apparatus shown in FIG. 1. The apparatus for manufacturing the olefin derivatives is equipped with a carbon monoxide (CO) manufacturing apparatus, a liquid nitrogen tank, an air introducing apparatus, a gas mixing apparatus, buffer tanks, mass flow controllers, a reactor, a carbon monoxide separating apparatus, and a vent apparatus.

The carbon monoxide manufacturing apparatus, the liquid nitrogen tank, and the air introducing apparatus are each connected to the gas mixing apparatus through a gas transporting pipe. The gas mixing apparatus is connected to the buffer tanks and the vent apparatus each through a gas transporting pipe. The buffer tanks are each connected to the reactor through a gas transporting pipe and the mass flow controller. The reactor is connected to the carbon monoxide separating apparatus through a gas transporting pipe and the mass flow controller. The carbon monoxide separating apparatus is connected to the gas mixing apparatus and the vent apparatus each through a gas transporting pipe.

In the carbon monoxide manufacturing apparatus, carbon monoxide is manufactured, for example, by steam reforming of methane (Cl). The carbon monoxide is supplied from the manufacturing apparatus to the gas mixing apparatus. Nitrogen is supplied from the liquid nitrogen tank to the gas mixing apparatus. Air is supplied from the air introducing apparatus to the gas mixing apparatus. In the gas mixing apparatus, carbon monoxide, nitrogen and air are mixed in a blend ratio adapted to the synthesis of olefin derivatives (carboxylic acids or carboxylate esters) to thereby prepare a mixed gas. The mixed gas is stored in the buffer tank, and supplied from the buffer tank to the reactor. The reactor may be equipped with a mechanism to heat its content and control its temperature. The reactor may further be equipped with a mechanism to stir its content. The reactor may be equipped with a mechanism to measure the pressure of each gas in its interior. The supply amount, the supply time and the supply timing of the mixed gas to the reactor are freely controlled by the buffer tank, the mass flow controller and the like. Here, a pure oxygen supplying apparatus may be connected to the gas mixing apparatus through a gas transporting pipe. The pure oxygen may be contained in the mixed gas as an oxidizing agent.

As the carbon monoxide separating apparatus, for example, an apparatus for carrying out a pressure swing adsorption (PSA) method may be used. The pressure swing adsorption method in the present embodiment is a method of allowing an adsorbent to adsorb carbon monoxide in the mixed gas of a high pressure and thereafter allowing the adsorbent to desorb carbon monoxide in an atmosphere of a low pressure to thereby separate carbon monoxide from the gas. The adsorbent has a function of selectively adsorbing carbon monoxide. The adsorbent has, for example, a porous carrier and a copper compound carried on the carrier. As the carrier, for example, at least one selected from the group consisting of alumina, zeolite, active carbon, silica and polystyrene resins may be used. As the copper compound, for example, a copper halide such as CuCl or CuCl₂ may be used.

The carbon monoxide separating apparatus is not limited to an apparatus for carrying out the pressure swing adsorption method. The carbon monoxide separating apparatus may be an apparatus for carrying out at least one separating method selected from the group consisting of a pressure swing adsorption method, a thermal swing adsorption (TSA) method (a temperature swing adsorption method), a temperature-pressure swing adsorption (TPSA) method, a membrane separation method and a cryogenic separation method. These carbon monoxide separating methods may be combined with each other. The thermal swing adsorption method is a method comprising a step of allowing an adsorbent to adsorb carbon monoxide in a mixed gas at a low temperature, and a step of heating the adsorbent to allow the adsorbent to desorb carbon monoxide. The temperature-pressure swing adsorption method is a method of allowing an adsorbent of a low temperature to adsorb carbon monoxide in a mixed gas of a high pressure, and thereafter allowing the adsorbent heated in a low-pressure atmosphere to desorb carbon monoxide. The membrane separation method is a method of selectively separating carbon monoxide from a mixed gas by using a gas permselective membrane. The cryogenic separation method is a method in which a mixed gas is cooled to low temperatures to liquefy the mixed gas, and carbon monoxide is separated and recovered by distillation or partial condensation utilizing differences in temperature when each gas condenses.

The method for manufacturing olefin derivatives according to the present embodiment comprises the following first step, second step and third step. The second step and the third step are carried out during the first step.

In the first step, a palladium catalyst and an oxidizing agent are introduced to a reactor. As required, a solvent may further be introduced to the reactor. The oxidizing agent has an activity of oxidizing and reproducing the palladium catalyst reduced during the reaction. Olefins and an alcohol being raw materials of carboxylic acids or carboxylate esters are further introduced to the reactor. The mixed gas containing oxygen and carbon monoxide is supplied to the reactor. Also oxygen in the mixed gas oxidizes and reproduces the palladium catalyst reduced during the reaction. The alcohol is not only a raw material but also functions as a solvent to dissolve the olefins and the carbon monoxide. In the reactor, the olefins are reacted with the alcohol and the carbon monoxide to thereby synthesize carboxylic acids or carboxylate esters.

In the second step, at least part of the mixed gas (residual gas in the reactor) in the reactor is discharged out of the reactor, and supplied to the carbon monoxide separating apparatus. In the separating apparatus, unreacted carbon monoxide is separated from the mixed gas. The carbon monoxide and the separated residual gas (nitrogen, oxygen and the like) may be discharged out of the manufacturing apparatus by the vent apparatus.

In the third step, carbon monoxide separated from the mixed gas in the separating apparatus is supplied to the reactor through the gas mixing apparatus, the buffer tank and the mass flow controller. In the third step, carbon monoxide separated from the mixed gas in the separating apparatus may be mixed with at least any one of air, nitrogen, and carbon monoxide from the carbon monoxide manufacturing apparatus in the gas mixing apparatus to thereby prepare a mixed gas, and the mixed gas may be supplied to the reactor. Alternatively, in the third step, only carbon monoxide separated from the mixed gas in the separating apparatus may be supplied from the separating apparatus directly to the reactor through a transporting pipe (and the mass flow controller) (not shown in figure).

The first step, the second step and the third step may be carried out simultaneously and parallelly. As long as the second step and the third step are carried out during the first step, the second step and the third step do not necessarily need to be carried out simultaneously and parallelly. During the first step, the second step may be carried out continuously, or may be carried out intermittently. During the first step, the third step may be carried out continuously, or may be carried out intermittently.

In the present embodiment, since carbon monoxide not having reacted with the olefins in the reactor is reutilized in the second step and the third step, the yield of the olefin derivatives is improved as compared with conventional manufacturing methods which waste unreacted carbon monoxide as residual gas. Further in the present embodiment, by the reutilization of carbon monoxide, wastefulness of carbon monoxide being a raw material can be reduced; the amount manufactured and the manufacturing cost of the carbon monoxide itself can be reduced; and the manufacturing efficiency of the olefin derivatives can be improved.

In the first step, the carbon monoxide dissolved in the solvent (including the alcohol) together with the alcohol react with the olefins to thereby produce carboxylic acids or carboxylate esters. Therefore, in order to enhance the production speed (manufacturing efficiency) of the carboxylic acids or the carboxylate esters, the dissolution of carbon monoxide in the solvent may be promoted. The solubility of each gas of carbon monoxide and others constituting the mixed gas in the reactor in the solvent increases along with a rise in the pressure of the each gas. The dissolution of carbon monoxide in the solvent, however, competes with the dissolution of gases other than carbon monoxide (for example, nitrogen) in the solvent. The present inventors consider that in conventional manufacturing methods, since the pressure of gases such as nitrogen accumulated without participating in the reaction in the reactor is high, the dissolution of nitrogen and the like in the solvent inhibits the dissolution of carbon monoxide in the solvent, and makes one cause of the decrease in the production speed of carboxylic acids or carboxylate esters. In the second step according to the present embodiment, however, gases not contributing to the reaction, such as nitrogen or reaction by-products in the reactor, are discharged out of the reactor. Hence, in the first step according to the present embodiment, the pressures of the gases not contributing to the reaction in the reactor are always regulated at low values to thereby enable to suppress the inhibition by these gases of the dissolution of carbon monoxide in the solvent. Further in the present embodiment, the third step enables the first step to always regulate the pressure of carbon monoxide in the reactor at an optimum value and promote the dissolution of carbon monoxide in the solvent. For these reasons, the present embodiment enables to always maintain the production speed of the carboxylic acids or the carboxylate esters in the first step at a high value, and to enhance the production efficiency thereof. Further in the present embodiment, the third step enables the first step to be continued without being discontinued until the entire reaction (reaction with carbon monoxide) of the olefins in the reactor is completed, and the yield can be improved.

In the present embodiment, when oxygen is used as the oxidizing agent of the palladium catalyst, the third step also can always replenish oxygen consumed for the oxidation (reproduction) of the palladium catalyst in the reactor in the first step, to the reactor. Therefore, in the present embodiment, the first step is enabled to always maintain the high activity of the palladium catalyst, to always maintain the production speed of the carboxylic acids or the carboxylate esters at a high value, and to enhance the manufacturing efficiency thereof.

In the present embodiment, since the second step and the third step are carried out continuously or intermittently during the first step, the manufacturing efficiency is enabled to be enhanced as compared with a batch-type manufacturing method in which the charging of a raw material gas to the pressure vessel, and the discharge of residual gas from the pressure vessel must be carried out for every one-time manufacture.

In the present embodiment, since the second step in which the residual gas in the reactor is discharged out of the reactor is carried out during the first step, the accumulation of the residual gas in the reactor can be suppressed and the atmospheric pressure in the reactor can always be maintained at a safe level.

It is preferable that the total pressure in the mixed gas in the reactor in the first step be 0.5 to 6 MPaG, and more preferable that the total pressure be 3 to 5 MPaG. It is preferable that the concentration of carbon monoxide in the mixed gas in the reactor be 15 to 70%, and more preferable that the concentration be 20 to 30%. Here, the concentration of carbon monoxide in the mixed gas refers to a proportion of a partial pressure of the carbon monoxide with respect to the total pressure of the mixed gas in the reactor. When oxygen is used as the oxidizing agent, although it is more preferable that the higher the concentration of oxygen is in the mixed gas in the reactor, a too high concentration thereof extends the explosion limit of carbon monoxide and resultantly decreases the safety of the manufacturing method. Therefore, the concentration of oxygen in the mixed gas in the reactor may be 4 to 8%. Here, the concentration of oxygen in the mixed gas is 0.21 times a proportion of a partial pressure of air with respect to the total pressure of the mixed gas in the reactor. These numerical values can be controlled freely by the gas mixing apparatus, the mass flow controller and the like. Here, even if the total pressure of the mixed gas, the concentration of carbon monoxide, and the concentration of oxygen are out of the above-mentioned ranges, the advantage of the present invention can be achieved.

It is preferable that the reaction temperature of the synthesis reaction of the carboxylic acids or the carboxylate esters in the first step be room temperature (25° C.) to 120° C., and it is more preferable that the temperature be 80 to 100° C. It is better to stir the raw material in the reactor in the first step. It is preferable that the stirring speed of the raw material be about 400 to 600 rpm. It is preferable that the concentration of the palladium catalyst in the liquid phase in which the carboxylic acids or the carboxylate esters are synthesized be regulated at 0.01 to 10% by mol, and it is more preferable that the concentration be 0.01 to 1.0% by mol. When a copper compound is used as the oxidizing agent, although it is more preferable that the higher the concentration of the copper compound is in the liquid phase, a too high concentration thereof makes a refining step of the carboxylic acids or the carboxylate esters to be complicated. Therefore, it is preferable to regulate the concentration of the copper compound in the liquid phase at 4 to 6% by mol. A chloride such as copper chloride may be added as an oxidation copromoter. Although it is more preferable that the higher the concentration of chlorine ions is in the liquid phase, a too high concentration thereof causes chlorine to corrode the reactor. Therefore, it is preferable that the concentration of chlorine ions in the liquid phase be regulated at 5 to 25% by mol. Here, even if the reaction temperature, the stirring speed, the concentration of the palladium catalyst, the concentration of the copper compound and the concentration of chlorine ions are out of the above-mentioned ranges, the advantage of the present invention can be achieved.

(Specific Aspect of Olefin Derivatives)

In the present embodiment, the olefins used as the raw material are not especially limited, but may be, for example alicyclic olefins. The alicyclic olefins are not especially limited, but may be, for example, norbornene-type compounds. Hereinafter, as an example of the method for manufacturing olefin derivatives according to the present invention, a method will be described which synthesizes norbornane-2-spiro-α-cycloalkanone-α′-spiro-2″-norbornane-5,5″,6,6″-tetracarboxylic acids and esters thereof from 5-norbornene-2-spiro-α-cycloalkanone-α′-spiro-2″-5″-norbornenes as one kind of alicyclic olefins. Precursors of alicyclic olefins, alicyclic carboxylic anhydrides, and polyimides obtained from the anhydrides will further be described.

### Anhydrides of norbornane-2-spiro-α-cycloalkanone-α′-spiro-2″-norbornane-5,5″,6,6″-tetracarboxylic acids

Norbornane-2-spiro-α-cycloalkanone-α′-spiro-2″-norbornane-5,5″,6,6″-tetracarboxylic dianhydrides manufactured in the present embodiment are represented by the following formula (1).

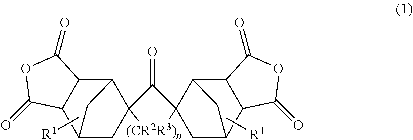

[In the formula (1), R¹, R² and R³ each independently represent one selected from the group consisting of a hydrogen atom, alkyl groups having a carbon number of 1 to 10, and a fluorine atom; and n represents an integer of 0 to 12.]

The norbornane-2-spiro-α-cycloalkanone-α′-spiro-2″-norbornane-5,5″,6,6″-tetracarboxylic dianhydrides are suitable as monomers of polyimides.

The alkyl group selectable as R¹ in the formula (1) is an alkyl group having a carbon number of 1 to 10. If the carbon number exceeds 10, when the carboxylic dianhydrides are used as monomers of polyimides, the heat resistance of the obtained polyimides decreases. From the viewpoint of providing a higher heat resistance when the polyimides are manufactured, it is preferable that the carbon number of the alkyl group selectable as R¹ be 1 to 6; it is more preferable that the carbon number be 1 to 5; it is still more preferable that the carbon number be 1 to 4; and it is especially preferable that the carbon number be 1 to 3. The alkyl group selectable as R¹ may be a straight chain one or a branched chain one.

It is more preferable from the viewpoint of providing a higher heat resistance when the polyimides are manufactured that R¹ in the formula (1) be each independently a hydrogen atom or an alkyl group having a carbon number of 1 to 10; and particularly from the viewpoint of the easiness of the availability of the raw material and the more easiness of the refinement, it is more preferable that the R¹ be a hydrogen atom, a methyl group, an ethyl group, a n-propyl group or an isopropyl group, and it is especially preferable that the R¹ be a hydrogen atom or a methyl group. It is especially preferable from the viewpoint of the easiness of the refinement and the like that the plurality of R¹ in the formula (1) be identical.

In the formula (1), n represents an integer of 0 to 12. If the value of n exceeds the upper limit, the refinement of norbornane-2-spiro-α-cycloalkanone-α′-spiro-2″-norbornane-5,5″,6,6″-tetracarboxylic dianhydrides becomes difficult. From the viewpoint of the more easiness of the refinement, it is more preferable that the upper limit of n in the formula (1) be 5, and it is especially preferable that the upper limit be 3. From the viewpoint of the stability of the raw material, and the like, it is more preferable that the lower limit of n in the formula (1) be 1, and it is especially preferable that the lower limit be 2. It is thus especially preferable that the integer n in the formula (1) be 2 to 3.

The alkyl group having 1 to 10 carbon atoms selectable as R² and R³ in the formula (1) is the same as the alkyl group having a carbon number of 1 to 10 selectable as R. From the viewpoint of the easiness of the refinement, it is preferable that a substituent selectable as R² and R among the above-mentioned substituents be a hydrogen atom or an alkyl group having a carbon number of 1 to 10, and it is especially preferable that the substituent be a hydrogen atom or a methyl group. The carbon number of the alkyl group is preferably 1 to 6, more preferably 1 to 5, still more preferably 1 to 4, and especially preferably 1 to 3.

Specific examples of the norbornane-2-spiro-α-cycloalkanone-α′-spiro-2″-norbornane-5,5″,6,6″-tetracarboxylic dianhydrides represented by the formula (1) include norbornane-2-spiro-α-cyclopentanone-α′-spiro-2″-norbornane-5,5″,6,6″-tetracarboxylic dianhydride (another nomenclature: norbornane-2-spiro-2′-cyclopentanone-5′-spiro-2″-norbornane-5,5″,6,6″-tetracarboxylic dianhydride), methylnorbornane-2-spiro-α-cyclopentanone-α′-spiro-2″-(methylnorbornane)-5,5″,6,6″-tetracarboxylic dianhydride, norbornane-2-spiro-α-cyclohexanone-α′-spiro-2″-norbornane-5,5″,6,6″-tetracarboxylic dianhydride (another nomenclature: norbornane-2-spiro-2′-cyclohexanone-6′-spiro-2″-norbornane-5,5″,6,6″-tetracarboxylic dianhydride), methylnorbornane-2-spiro-α-cyclohexanone-α′-spiro-2″-(methylnorbornane)-5,5″,6,6″-tetracarboxylic dianhydride, norbornane-2-spiro-α-cyclopropanone-α-spiro-2″-norbornane-5,5″,6,6″-tetracarboxylic dianhydride, norbornane-2-spiro-α-cyclobutanone-α′-spiro-2″-norbornane-5,5″,6,6″-tetracarboxylic dianhydride, norbornane-2-spiro-α-cycloheptanone-α′-spiro-2″-norbornane-5,5″,6,6″-tetracarboxylic dianhydride, norbornane-2-spiro-α-cyclooctanone-α′-spiro-2″-norbornane-5,5″,6,6″-tetracarboxylic dianhydride, norbornane-2-spiro-α-cyclononanone-α′-spiro-2″-norbornane-5,5″,6,6″-tetracarboxylic dianhydride, norbornane-2-spiro-α-cyclodecanone-α′-spiro-2″-norbornane-5,5″,6,6″-tetracarboxylic dianhydride, norbornane-2-spiro-α-cycloundecanone-α′-spiro-2″-norbornane-5,5″,6,6″-tetracarboxylic dianhydride, norbornane-2-spiro-α-cyclododecanone-α′-spiro-2″-norbornane-5,5″,6,6″-tetracarboxylic dianhydride, norbornane-2-spiro-α-cyclotridecanone-α′-spiro-2″-norbornane-5,5″,6,6″-tetracarboxylic dianhydride, norbornane-2-spiro-α-cyclotetradecanone-α′-spiro-2″-norbornane-5,5″,6,6″-tetracarboxylic dianhydride, norbornane-2-spiro-α-cyclopentadecanone-α′-spiro-2″-norbornane-5,5″,6,6″-tetracarboxylic dianhydride, norbornane-2-spiro-α-(methylcyclopentanone)-α′-spiro-2″-norbornane-5,5″,6,6″-tetracarboxylic dianhydride, and norbornane-2-spiro-α-(methylcyclohexanone)-α′-spiro-2″-norbornane-5,5″,6,6″-tetracarboxylic dianhydride.

### Norbornane-2-spiro-α-cycloalkanone-α′-spiro-2″-norbornane-5,5″,6,6″-carboxylic acids and esters thereof

Norbornane-2-spiro-α-cycloalkanone-α′-spiro-2″-norbornane-5,5″,6,6″-tetracarboxylic acids and esters thereof in the present embodiment are represented by the following formula (2).

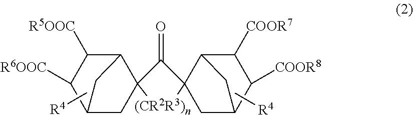

[In the formula (2), R², R³ and R⁴ each independently represent one selected from the group consisting of a hydrogen atom, alkyl groups having a carbon number of 1 to 10, and a fluorine atom; R⁵, R⁶, R⁷ and R⁸ each independently represent one selected from the group consisting of a hydrogen atom, alkyl groups having a carbon number of 1 to 10, cycloalkyl groups having a carbon number of 3 to 10, alkenyl groups having a carbon number of 2 to 10, aryl groups having a carbon number of 6 to 20, and aralkyl groups having a carbon number of 7 to 20; and n represents an integer of 0 to 12.]

R⁴ in the formula (2) are the same as R¹ in the above formula (1), and suitable R⁴ are also the same as R¹ in the above formula (1). R² and R³ in the formula (2) are the same as R² and R³ in the formula (1), and suitable R² and R³ are also the same as R² and R³ in the above formula (1). Further n in the above formula (2) is the same integer as n in the above formula (1), and its suitable value is also the same as n in the above formula (1).

The alkyl group selectable as R⁵, R⁶, R⁷ and R⁸ in the formula (2) is an alkyl group having a carbon number of 1 to 10. If the carbon number of the alkyl group exceeds 10, the refinement becomes difficult. From the viewpoint of the more easiness of the refinement, it is more preferable that the carbon number of the alkyl group selectable as R⁵, R⁶, R⁷ and R⁸ be 1 to 5; and it is still more preferable that the carbon number be 1 to 3. The alkyl group selectable as R⁵, R⁶, R⁷ and R⁸ may be a straight chain one or a branched chain one.

The cycloalkyl group selectable as R⁵, R⁶, R⁷ and R⁸ in the formula (2) is a cycloalkyl group having 3 to 10 carbon atoms. If the carbon number of the cycloalkyl group exceeds 10, the refinement becomes difficult. From the viewpoint of the more easiness of the refinement, it is more preferable that the carbon number of such a cycloalkyl group selectable as R⁵, R⁶, R⁷ and R⁸ be 3 to 8; and it is still more preferable that the carbon number be 5 to 6.

The alkenyl group selectable as R⁵, R⁶, R⁷ and R⁸ in the formula (2) is an alkenyl group having a carbon number of 2 to 10. If the carbon number of the alkenyl group exceeds 10, the refinement becomes difficult. From the viewpoint of the more easiness of the refinement, it is more preferable that the carbon number of the alkenyl group selectable as R⁵, R⁶, R⁷ and R⁸ be 2 to 5; and it is still more preferable that the carbon number be 2 to 3.

The aryl group selectable as R⁵, R⁶, R⁷ and R⁸ in the formula (2) is an aryl group having a carbon number of 6 to 20. If the carbon number of the aryl group exceeds 20, the refinement becomes difficult. From the viewpoint of the more easiness of the refinement, it is more preferable that the carbon number of the aryl group selectable as R⁵, R⁶, R⁷ and R⁸ be 6 to 10; and it is still more preferable that the carbon number be 6 to 8.

The aralkyl group selectable as R⁵, R⁶, R⁷ and R⁸ in the formula (2) is an aralkyl group having a carbon number of 7 to 20. If the carbon number of the aralkyl group exceeds 20, the refinement becomes difficult. From the viewpoint of the more easiness of the refinement, it is more preferable that the carbon number of the aralkyl group selectable as R⁵, R⁶, R⁷ and R⁸ be 7 to 10; and it is still more preferable that the carbon number be 7 to 9.

From the viewpoint of the more easiness of the refinement, it is preferable that R⁵, R⁶, R⁷ and R⁸ in the formula (2) be each independently a hydrogen atom, a methyl group, an ethyl group, a n-propyl group, an isopropyl group, a n-butyl group, an isobutyl group, a sec-butyl group, a t-butyl group, a 2-ethylhexyl group, a cyclohexyl group, an allyl group, a phenyl group or a benzyl group; and it is especially preferable that these be a methyl group. Here, R⁵, R⁶, R⁷ and R⁸ in the formula (2) may be identical or different, but it is more preferable that these be identical from the viewpoint of the synthesis.

Specific examples of norbornane-2-spiro-α-cycloalkanone-α′-spiro-2″-norbornane-5,5″,6,6″-tetracarboxylic acids and esters thereof represented by the formula (2) include norbornane-2-spiro-α-cyclopentanone-α′-spiro-2″-norbornane-5,5″,6,6″-tetracarboxylic acid tetramethyl ester, norbornane-2-spiro-α-cyclopentanone-α′-spiro-2″-norbornane-5,5″,6,6″-tetracarboxylic acid tetraethyl ester, norbornane-2-spiro-α-cyclopentanone-α′-spiro-2″-norbornane-5,5″,6,6″-tetracarboxylic acid tetrapropyl ester, norbornane-2-spiro-α-cyclopentanone-α′-spiro-2″-norbornane-5,5″,6,6″-tetracarboxylic acid tetrabutyl ester, norbornane-2-spiro-α-cyclopentanone-α′-spiro-2″-norbornane-5,5″,6,6″-tetracarboxylic acid tetra(2-ethylhexyl) ester, norbornane-2-spiro-α-cyclopentanone-α′-spiro-2″-norbornane-5,5″,6,6″-tetracarboxylic acid tetraallyl ester, norbornane-2-spiro-α-cyclopentanone-α′-spiro-2″-norbornane-5,5″,6,6″-tetracarboxylic acid tetracyclohexyl ester, norbornane-2-spiro-α-cyclopentanone-α′-spiro-2″-norbornane-5,5″,6,6″-tetracarboxylic acid tetraphenyl ester, norbornane-2-spiro-α-cyclopentanone-α′-spiro-2″-norbornane-5,5″,6,6″-tetracarboxylic acid tetrabenzyl ester, norbornane-2-spiro-α-cyclopentanone-α′-spiro-2″-norbornane-5,5″,6,6″-tetracarboxylic acid, methylnorbornane-2-spiro-α-cyclopentanone-α′-spiro-2″-(methylnorbornane)-5,5,6,6″-tetracarboxylic acid tetramethyl ester, norbornane-2-spiro-α-cyclohexanone-α′-spiro-2″-norbornane-5,5″,6,6″-tetracarboxylic acid tetramethyl ester, norbornane-2-spiro-α-cyclohexanone-α′-spiro-2″-norbornane-5,5″,6,6″-tetracarboxylic acid tetraethyl ester, norbornane-2-spiro-α-cyclohexanone-α′-spiro-2″-norbornane-5,5″,6,6″-tetracarboxylic acid tetrapropyl ester, norbornane-2-spiro-α-cyclohexanone-α′-spiro-2″-norbornane-5,5″,6,6″-tetracarboxylic acid tetrabutyl ester, norbornane-2-spiro-α-cyclohexanone-α′-spiro-2″-norbornane-5,5″,6,6″-tetracarboxylic acid tetra(2-ethylhexyl) ester, norbornane-2-spiro-α-cyclohexanone-α′-spiro-2″-norbornane-5,5″,6,6″-tetracarboxylic acid tetraallyl ester, norbornane-2-spiro-α-cyclohexanone-α′-spiro-2″-norbornane-5,5″,6,6″-tetracarboxylic acid tetracyclohexyl ester, norbornane-2-spiro-α-cyclohexanone-α′-spiro-2″-norbornane-5,5″,6,6″-tetracarboxylic acid tetraphenyl ester, norbornane-2-spiro-α-cyclohexanone-α′-spiro-2″-norbornane-5,5″,6,6″-tetracarboxylic acid tetrabenzyl ester, norbornane-2-spiro-α-cyclohexanone-α′-spiro-2″-norbornane-5,5″,6,6″-tetracarboxylic acid, methylnorbornane-2-spiro-α-cyclohexanone-α′-spiro-2″-(methylnorbornane)-5,5″,6,6″-tetracarboxylic acid tetramethyl ester, norbornane-2-spiro-α-cyclopropanone-α′-spiro-2″-norbornane-5,5″,6,6″-tetracarboxylic acid tetramethyl ester, norbornane-2-spiro-α-cyclobutanone-α′-spiro-2″-norbornane-5,5″,6,6″-tetracarboxylic acid tetramethyl ester, norbornane-2-spiro-α-cycloheptanone-α′-spiro-2″-norbornane-5,5″,6,6″-tetracarboxylic acid tetramethyl ester, norbornane-2-spiro-α-cyclooctanone-α′-spiro-2″-norbornane-5,5″,6,6″-tetracarboxylic acid tetramethyl ester, norbornane-2-spiro-α-cyclononanone-α′-spiro-2″-norbornane-5,5″,6,6″-tetracarboxylic acid tetramethyl ester, norbornane-2-spiro-α-cyclodecanone-α′-spiro-2″-norbornane-5,5″,6,6″-tetracarboxylic acid tetramethyl ester, norbornane-2-spiro-α-cycloundecanone-α′-spiro-2″-norbornane-5,5″,6,6″-tetracarboxylic acid tetramethyl ester, norbornane-2-spiro-α-cyclododecanone-α′-spiro-2″-norbornane-5,5″,6,6″-tetracarboxylic acid tetramethyl ester, norbornane-2-spiro-α-cyclotridecanone-α′-spiro-2″-norbornane-5,5″,6,6″-tetracarboxylic acid tetramethyl ester, norbornane-2-spiro-α-cyclotetradecanone-α′-spiro-2″-norbornane-5,5″,6,6″-tetracarboxylic acid tetramethyl ester, norbornane-2-spiro-α-cyclopentadecanon-ε-spiro-2″-norbornane-5,5″,6,6″-tetracarboxylic acid tetramethyl ester, norbornane-2-spiro-α-cyclopentanone-α′-spiro-2″-norbornane-5,5″,6,6″-tetracarboxylic acid, and norbornane-2-spiro-α-cyclohexanone-α′-spiro-2″-norbornane-5,5″,6,6″-tetracarboxylic acid.

### Method for synthesizing norbornane-2-spiro-α-cycloalkanone-α′-spiro-2″-norbornane-5,5″,6,6″-tetracarboxylic acids or esters thereof

A synthesizing method (first step) of norbornane-2-spiro-α-cycloalkanone-α′-spiro-2′-norbornane-5,5″,6,6″-tetracarboxylic acids or esters thereof represented by the above formula (2) in the present embodiment will be described.

In the first step, 5-norbornene-2-spiro-α-cycloalkanone-α′-spiro-2″-5″-norbornenes represented by the following formula (3) are reacted with an alcohol and carbon monoxide in the presence of a palladium catalyst and an oxidizing agent to thereby obtain compounds represented by the above formula (2).

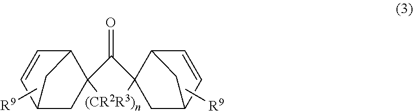

[In the formula (3), R², R³ and R⁹ each independently represent one selected from the group consisting of a hydrogen atom, alkyl groups having a carbon number of 1 to 10, and a fluorine atom; and n represents an integer of 0 to 12.]

In norbornenes represented by the above formula (3) used in the first step, R⁹ are the same as R¹ in the above formula (1), and suitable R⁹ are also the same as R¹ in the above formula (1). R² and R³ in the formula (3) are the same as R² and R³ in the formula (1), and suitable R² and R³ are also the same as R² and R³ in the above formula (1). Further n in the above formula (3) is the same integer as n in the above formula (1), and its suitable value is also the same as n in the above formula (1).

Specific examples of the compounds represented by the formula (3) include 5-norbornene-2-spiro-α-cyclopentanone-α′-spiro-2″-5″-norbornene (another nomenclature: 5-norbornene-2-spiro-2′-cyclopentanone-5′-spiro-2″-5″-norbornene), methyl-5-norbornene-2-spiro-α-cyclopentanone-α′-spiro-2″-(methyl-5″-norbornene), 5-norbornene-2-spiro-α-cyclohexanone-α′-spiro-2″-5″-norbornene (another nomenclature: 5-norbornene-2-spiro-2′-cyclohexanone-6′-spiro-2″-5″-norbornene), methyl-5-norbornene-2-spiro-α-cyclohexanone-α′-spiro-2″-(methyl-5″-norbornene), 5-norbornene-2-spiro-α-cyclopropanone-α′-spiro-2″-5″-norbornene, 5-norbornene-2-spiro-α-cyclobutanone-α′-spiro-2″-5″-norbornene, 5-norbornene-2-spiro-α-cycloheptanone-α′-spiro-2″-5″-norbornene, 5-norbornene-2-spiro-α-cyclooctanone-α′-spiro-2″-5″-norbornene, 5-norbornene-2-spiro-α-cyclononanone-α′-spiro-2″-5″-norbornene, 5-norbornene-2-spiro-α-cyclodecanone-α′-spiro-2″-5″-norbornene, 5-norbornene-2-spiro-α-cycloundecanone-α′-spiro-2″-5″-norbornene, 5-norbornene-2-spiro-α-cyclododecanone-α′-spiro-2″-5″-norbornene, 5-norbornene-2-spiro-α-cyclotridecanone-α′-spiro-2″-5″-norbornene, 5-norbornene-2-spiro-α-cyclotetradecanone-α′-spiro-2″-5″-norbornene, 5-norbornene-2-spiro-α-cyclopentadecanone-α′-spiro-2″-5″-norbornene, 5-norbornene-2-spiro-α-(methylcyclopentanone)-α′-spiro-2″-5″-norbornene, and 5-norbornene-2-spiro-α-(methylcyclohexanone)-α′-spiro-2″-5″-norbornene.

A method for synthesizing compounds represented by the formula (3) is not especially limited, but the compounds represented by the formula (3) can be synthesized, for example, by utilizing a reaction represented by the following reaction formula (I).

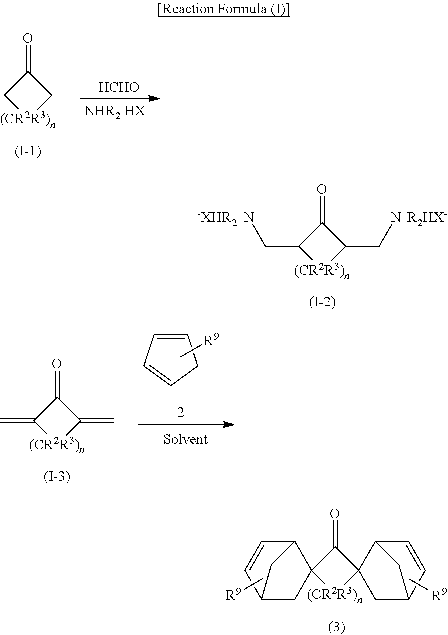

[n R² and R³ in the reaction formula (I) are the same as n, R² and R³ in the above formula (1); R⁹ in the reaction formula (I) are the same as R⁹ in the above formula (3); R in the reaction formula (I) each independently represent a monovalent organic group (for example, a straight chain saturated hydrocarbon group having a carbon number of 1 to 20) capable of forming an amine; and X⁻ in the reaction formula (I) represents a monovalent ion (for example, a halogen ion, a hydrogensulfate ion or an acetate ion) capable of forming an ammonium salt with an amine.]

In the synthesizing method represented by the reaction formula (I), cycloalkanones (cyclopentanone, cyclohexanone and the like) represented by the formula (I-1), ammonium salts of secondary amines (for example, a hydrochloride salt, a sulfate salt or an acetate salt: a compound represented by the formula: NHR₂HX in the reaction formula (I)) in an amount of not less than 2 equivalents with respect to the cycloalkanones, formaldehyde derivatives, and an acid (hydrochloric acid, sulfuric acid, acetic acid or the like) are used to thereby obtain an acidic reaction solution. The reaction solution is heated in an inert gas atmosphere at 30 to 180° C. for 0.5 to 10 hours to cause Mannich reaction of the cyclic ketones both neighbors of whose carbonyl groups have active ac hydrogen, the formaldehydes and the secondary amines to proceed in the reaction solution, to thereby synthesize Mannich bases represented by the formula (I-2). Then, without the obtained Mannich bases being isolated, an organic solvent and a cyclopentadiene (in an amount of not less than 2 equivalents with respect to the Mannich bases) which may have, as a substituent, the same group as the group selectable as R¹ in the above formula (1) are added to the reaction solution to thereby prepare a mixture. The organic solvent may be an organic solvent capable of being utilized in Diels-Alder reaction; and there may preferably be used tetrahydrofuran, methanol, ethanol, isopropanol, butanol, acetonitrile, methyl cellosolve, ethyl cellosolve, ethylene glycol, propylene glycol monomethyl ether, propylene glycol or the like. A base is introduced to the mixture to thereby make the mixture to be neutral or basic, and the mixture is stirred under the condition of 0 to 150° C. (preferably about 60° C.) for 0.1 to 48 hours. By this operation, divinyl ketones represented by the formula (I-3) are synthesized from the Mannich bases represented by the formula (I-2) in the mixture. By the reaction (Diels-Alder reaction) of the divinyl ketones represented by the formula (I-3) with the above cyclopentadiene which may have the substituent, the compounds represented by the above formula (3) can be synthesized. Here, as the formaldehyde derivatives, well-known formaldehyde derivatives used in manufacture of Mannich bases can suitably be used, and examples thereof include formalin, paraformaldehyde, trioxane and 1,3-dioxolan. The vinyl ketones are synthesized by allowing the amine compounds to be eliminated from the Mannich bases during stirring under the condition of 0 to 150° C. of the mixture.

Specific examples of the cycloalkanones represented by the formula (I-1) in the reaction formula (I) include cyclopropanone, cyclobutanone, cyclopentanone, cyclohexanone, cycloheptanone, cyclooctanone, cyclononanone, cyclodecanone, cycloundecanone, cyclododecanone, cyclotridecanone, cyclotetradecanone, cyclopentadecanone, 3-methylcyclobutanone, 3-methylcyclopentanone, 3-methylcyclohexanone, 3-methylcycloheptanone, 3-methylcyclooctanone, 3-methylcyclononanone, 3-methylcyclodecanone, 3-methylcycloundecanone, 3-methylcyclododecanone, 3-methylcyclotridecanone, 3-methylcyclotetradecanone and 3-methylcyclopentadecanone. Specific examples of the ammonium salts of secondary amines include salts of the secondary amines (salts of the secondary amines in which the above X⁻ becomes a counter anion), such as dimethylamine, diethylamine, di-n-propylamine, diisopropylamine, di-n-butylamine, diisobutylamine, di-sec-butylamine, di-t-butylamine, dipentylamine, dicyclopentyl amine, dihexylamine, dicyclohexylamine, diheptylamine, dioctylamine, di(2-ethylhexyl)amine, dinonylamine, didecylamine, diundecylamine, didodecylamine, ditridecylamine, ditetradecylamine, dipentadecylamine, dihexadecylamine, diheptadecylamine, dioctadecylamine, dinonadecylamine, morpholine, diethanolamine, aziridine, azetidine, pyrrolidine, piperidine, indoline and isoindoline. In the reaction formula (I), X⁻ is a so-called counter anion. Specific examples of X⁻ include F⁻, Cl⁻, Br⁻, I⁻, CH₃COO⁻, CF₃COO⁻, CH₃SO₃⁻, CF₃SO₃⁻, C₆H₅SO₃⁻, CH₃C₆H₄SO₃⁻, HOSO₃⁻ and H₂PO₄⁻. The vinyl ketones are synthesized by allowing the amine compounds to be eliminated from the Mannich bases during stirring under the condition of 0 to 150° C. of the mixture.

It is preferable that the alcohol used in the first step be an alcohol represented by the following formula (5).

R¹¹OH (5)

[In the formula (5), R¹¹ is an atom or a group excluding a hydrogen atom among the atoms and groups selectable as R⁵, R⁶, R⁷ and R⁸ in the above formula (2).]

It is preferable that as the alcohol represented by the formula (5), there be used an alkyl alcohol having a carbon number of 1 to 10, a cycloalkyl alcohol having a carbon number of 3 to 10, an alkenyl alcohol having a carbon number of 2 to 10, an aryl alcohol having a carbon number of 6 to 20, or an aralkyl alcohol having a carbon number of 7 to 20. Specific examples of such an alcohol include methanol, ethanol, butanol, allyl alcohol, cyclohexanol and benzyl alcohol, and among these, methanol or ethanol are more preferable, and methanol is especially preferable, from the viewpoint of the more easiness of the refinement of compounds to be obtained. The alcohol may be used singly or as a mixture of two or more.

In the first step using the above alcohol, by reacting the alcohol (R¹¹OH) and carbon monoxide (CO) with compounds represented by the formula (3) in the presence of a palladium catalyst and an oxidizing agent, ester groups represented by the following formula (6) may be introduced to respective olefin sites in the compounds represented by the above formula (3). R¹¹ of all the ester groups may be identical. R¹¹ may be different for each position to which an ester group is introduced.

—COOR¹¹ (6)

[In the formula (6), R¹¹ is an atom or a group excluding a hydrogen atom among the atoms and groups selectable as R⁵, R⁶, R⁷ and R⁸ in the above formula (2).]

By such a reaction (esterification reaction), norbornane-2-spiro-α-cycloalkanone-α′-spiro-2″-norbornane-5,5″,6,6″-tetracarboxylate esters represented by the formula (2) are synthesized.

The amount of an alcohol used in the above esterification reaction may be an amount capable of providing compounds represented by the formula (2), and is not especially limited. For example, an amount (theoretical amount) of the alcohol theoretically required in order to provide the compounds represented by the formula (2), or a more amount thereof is supplied to the reaction system, and the surplus alcohol therefrom may be made to function as a solvent.

In the esterification reaction, an amount of carbon monoxide to be required may be supplied to the reaction system. Therefore, a high-purity gas of carbon monoxide does not need to be used.

The palladium catalyst used in the first step is not especially limited, and a well-known catalyst containing palladium can suitably be used. Specific examples of the palladium catalyst include inorganic acid salts of palladium, organic acid salts of palladium, and catalysts having a carrier carrying palladium. The palladium catalyst more specifically includes palladium chloride, palladium nitrate, palladium sulfate, palladium acetate, palladium propionate, palladium carbon, palladium alumina and palladium black. It is preferable that the amount of these palladium catalysts used be such that the molar quantity of palladium in the palladium catalysts is 0.001 to 0.1 times mole the mole of compounds represented by the above formula (3).

The oxidizing agent to be used in the first step may be one which, when Pd²⁺ is reduced to Pd⁰ in the palladium catalyst in the esterification reaction, is capable of oxidizing the Pd⁰ to Pd²⁺, and the oxidizing agent is not especially limited. Specific examples of the oxidizing agent include oxygen, copper compounds and iron compounds. The oxidizing agent more specifically includes oxygen, cupric acetate, cupric chloride, cupric nitrate, cupric sulfate, ferric chloride, ferric nitrate, ferric sulfate, and ferric acetate. It is preferable that the amount of these oxidizing agents used is an amount of 2 to 16 times mole (more preferably about 8 times mole) the mole of 5-norbornene-2-spiro-α-cycloalkanone-α′-spiro-2″-5″-norbornenes represented by the formula (3).

It is preferable that the reaction (esterification reaction) of the compounds represented by the formula (3) with the alcohol and carbon monoxide be carried out in a solvent. The solvent is not especially limited, and examples thereof include hydrocarbon-based solvents such as n-hexane, cyclohexane, heptane and pentane.

In the esterification reaction, since an acid is by-produced from the oxidizing agent and the like, a base may be added in order to remove such an acid. As such a base, fatty acid salts such as sodium acetate, sodium propionate and sodium butyrate are preferable. The amount of these bases used may suitably be regulated according to the amount of the acid generated, and the like.

In order to make R⁵, R⁶, R⁷ or R⁸ in the formula (2) to be a hydrogen atom, after the group represented by the above formula: —COOR¹¹ is introduced by the esterification reaction, a hydrolysis treatment or a transesterification reaction with a carboxylic acid may be carried out. A method of such a reaction is not especially limited, and a well-known method can suitably be used which is capable of turning the group represented by the formula: —COOR¹¹ to the formula: —COOH.

After the above esterification reaction, hydrolysis or the like is carried out, in order to provide higher-purity compounds, a refining step such as recrystallization may suitably be carried out. Such a refining method is not especially limited, and a well-known method can suitably be employed.

### A method for synthesizing norbornane-2-spiro-α-cycloalkanone-α′-spiro-2″-norbornane-5,5″,6,6″-tetracarboxylic dianhydrides

A synthesizing method (a fourth step) of norbornane-2-spiro-α-cycloalkanone-α′-spiro-2″-norbornane-5,5″,6,6″-tetracarboxylic dianhydrides represented by the above formula (1) will be described. In the fourth step, compounds represented by the above formula (1) are obtained from compounds represented by the above formula (2) by using formic acid, an acid catalyst and acetic anhydride.

In another aspect of the fourth step, the compounds represented by the above formula (2) synthesized in the first step are hydrolyzed in the presence of an acid catalyst or a base catalyst to thereby manufacture norbornane-2-spiro-α-cycloalkanone-α′-spiro-2″-norbornane-5,5″,6,6″-tetracarboxylic acids. By cyclodehydrating these by heating or with a dehydrating agent, the anhydrides represented by the above formula (1) are obtained.

<Polyimides>

The compounds represented by the above formula (1) are especially useful as a raw material of polyamic acids and a raw material of heat-resistant resins such as polyimides. An example of a method for synthesizing polyimides includes a method in which anhydrides represented by the above formula (1) are reacted with diamine compounds in a solvent to thereby obtain polyamic acids, and thereafter, the polyamic acids are cyclodehydrated by being heated or with an acid anhydride. Polyimides thus obtained have the compounds represented by the above formula (1) as one of monomers.

The polyimides according to the present embodiment have a sufficiently high-degree solvent solubility and are simultaneously transparent and colorless in spite of using aliphatic tetracarboxylic dianhydrides. The polyimides according to the present embodiment further have a higher heat resistance (a high glass transition temperature Tg) as compared with conventional polyimides fabricated from aliphatic tetracarboxylic dianhydrides. Therefore, the compounds represented by the above formula (1) according to the present embodiment are especially useful as a raw material for polyimides for flexible wiring boards, polyimides for heat-resistant insulating tapes, polyimides for electric wire enamels, polyimides for protective coatings for semiconductors, polyimides for liquid crystal alignment films, and the like.

The polyimides according to the present embodiment have a repeating unit represented by the following formula (4).

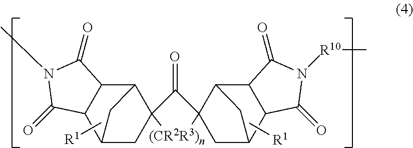

[In the formula (4), R¹, R² and R³ each independently represent one selected from the group consisting of a hydrogen atom, alkyl groups having a carbon number of 1 to 10, and a fluorine atom; R¹⁰ represents an aryl group having a carbon number of 6 to 40; and n represents an integer of 0 to 12.]

The above polyimides, since being obtained using alicyclic tetracarboxylic dianhydrides, are very high in transparency. Such polyimides are especially useful as a raw material for films for flexible wiring boards, heat-resistant insulating tapes, electric wire enamels, protective coating agents for semiconductors, liquid crystal alignment films, transparent electroconductive films for organic EL, flexible board films, flexible transparent electroconductive films, transparent electroconductive films for organic thin film-type solar cells, transparent electroconductive films for dye-sensitized solar cells, flexible gas barrier films, films for touch panels, and the like.

Hitherto, one preferred embodiment according to the present invention has been described in detail, but the present invention is not limited to the above embodiment.

### INDUSTRIAL APPLICABILITY

According to the present invention, the yield and manufacturing efficiency of olefin derivatives can be improved. The olefin derivatives obtainable by the present invention are used for manufacture of, for example, polyimides. The polyimides are used as a raw material for films for flexible wiring boards, heat-resistant insulating tapes, electric wire enamels, protective coating agents for semiconductors, liquid crystal alignment films, transparent electroconductive films for organic EL, flexible board films, flexible transparent electroconductive films, transparent electroconductive films for organic thin film-type solar cells, transparent electroconductive films for dye-sensitized solar cells, flexible gas barrier films, films for touch panels, and the like.

1. A method for manufacturing an olefin derivative, comprising:
a first step of reacting an olefin with an alcohol and carbon monoxide in the presence of a palladium catalyst and an oxidizing agent in a reactor to thereby synthesize a carboxylic acid or a carboxylate ester,
a second step of discharging at least part of a gas in the reactor out of the reactor, and separating carbon monoxide from the gas discharged during the first step; and
a third step of supplying the carbon monoxide separated from the gas in the second step to the reactor during the first step.

2. The method for manufacturing an olefin derivative according to claim 1, wherein the olefin is an alicyclic olefin.

3. The method for manufacturing an olefin derivative according to claim 2, wherein the alicyclic olefin is a norbornene-type compound.

4. The method for manufacturing an olefin derivative according to claim 1, wherein the gas discharged out of the reactor in the second step contains nitrogen.

5. The method for manufacturing an olefin derivative according to claim 1, wherein the carbon monoxide is separated from the gas by using at least one method selected from the group consisting of a pressure swing adsorption method, a thermal swing adsorption method, a temperature-pressure swing adsorption method, a membrane separation method and a cryogenic separation method.

6. The method for manufacturing an olefin derivative according to claim 1, wherein at least one selected from the group consisting of oxygen, cupric acetate, cupric chloride, cupric nitrate, cupric sulfate, ferric chloride, ferric nitrate, ferric sulfate and ferric acetate is used as the oxidizing agent.
